(19) United States
(12) Patent Application Publication
(10) Pub. No.: US 2024/0406125 A1
(43) Pub. Date: Dec. 5, 2024

(54) COMMUNICATION WITH PLANT-ASSOCIATED PERSONAS VIA ARTIFICIAL INTELLIGENCE

(71) Applicant: ePlant, Inc., Los Altos, CA (US)

(72) Inventors: Roger George HINE, Portola Valley, CA (US); Jesse Thomas, Los Altos, CA (US); Graham Lancaster HINE, Los Gatos, CA (US); Evan T. DELLOR, La Honda, CA (US); Kevin Hugh REA, Menlo Park, CA (US)

(73) Assignee: ePlant, Inc., Los Altos, CA (US)

(21) Appl. No.: 18/731,053

(22) Filed: May 31, 2024

Related U.S. Application Data
(60) Provisional application No. 63/505,633, filed on Jun. 1, 2023.

(51) Int. Cl. H04L 51/02; G06F 16/332

(52) U.S. Cl. CPC H04L 51/02; G06F 16/3329

(57) ABSTRACT

Described herein are systems and methods for monitoring plant health by communicating with plant-associated persona(s) via artificial intelligence. These systems and methods can communicate information about the health of plants in a natural-language format through the use of plant-based sensors for collecting data on plant health, analytical models and processors for processing the data on plant health, large language models for converting the data on plant health into a persona-guided, natural-language format, and user devices for receiving the data on plant health in the natural-language format.

### CROSS-REFERENCE TO RELATED APPLICATIONS

This application claims the benefit of U.S. Provisional Application No. 63/505,633, filed Jun. 1, 2023, which is hereby incorporated by reference in its entirety.

### FIELD

The present disclosure relates generally to communicating information about the health of plants, and more specifically to communicating with plant-associated persona(s) via artificial intelligence to receive information about the health of plants in a natural-language format.

### BACKGROUND

Monitoring the health of plants is a necessary part of maintaining a safe and healthy yard, farm, homestead, forest, or other plant-covered area. Typically, plant health monitoring relies on visual inspection by experts who can identify the presence of pests, diseases, or other stressors, based on qualitative symptoms. In addition to visual inspection, plant-based sensors, such as dendrometers, can be used to obtain quantitative data about the health of plants. However, these methods of plant health monitoring are only accessible to experts who have substantial experience in identifying symptoms or interpreting sensor data—for example, a new homeowner, wanting to monitor the health of unfamiliar trees on his or her property, would have difficulty performing these monitoring methods with confidence. Furthermore, these monitoring methods involve significant effort, physical proximity to the plants (to perform visual inspections or sensor readings), and a high likelihood of error when performed by both experts and non-experts. These are additional deterrents that prevent individuals from properly monitoring the health of their plants.

As such, there is a need for improved systems and methods for monitoring plant health that are accessible to both expert and non-expert users, can be performed remotely, and have a high degree of confidence. Such improved systems and methods will be able to process and communicate information about the health of plants in an easy-to-understand format, such as a natural-language format, allowing non-expert users to make educated decisions regarding the caretaking of their plants.

### BRIEF SUMMARY

Provided herein, inter alia, are systems and methods for monitoring plant health by communicating with plant-associated persona(s) via artificial intelligence. Specifically, the systems and methods provided herein can communicate information about the health of plants in a natural-language format by using plant-based sensors for collecting data on plant health, processors and analytical models for processing the data on plant health, large language models for converting the data on plant health into a persona-guided, natural-language format, and user devices for receiving the data on plant health in the natural-language format. Also provided herein are computer-program products (tangibly embodied in a non-transitory machine-readable storage medium) which include instructions associated with the systems and methods provided herein.

These systems and methods provide multiple technical advantages over existing plant health monitoring techniques, such as enabling non-expert users to understand relevant information about the health of their plants and to make educated decisions regarding the caretaking of their plants. Any user, regardless of his or her experience with plant health monitoring, can access these systems and methods through an application on a user device. This has the additional benefit of enabling the user to interact with plant-associated persona(s) remotely, without requiring physical proximity to the plant. Furthermore, because the processing of plant health data is performed by a trained analytical model and does not rely on the user's expertise, the systems and methods provided herein can have a higher degree of confidence than existing plant health monitoring techniques. In some instances, analytical model(s) about plant health and various plant parameter can be improved by the systems and methods of the present disclosure, e.g., by soliciting user input/feedback and updating the analytical model(s) in response, particularly when measurement(s) associated with the plant (e.g., from an associated sensor of the present disclosure) deviate from the analytical model(s).

According to some embodiments, provided herein is a method for providing a natural-language interface for conversing with a persona associated with a plant, comprising, at one or more processors: receiving a set of sensor data associated with the plant; generating, via an analytical model, a natural-language output based on the set of sensor data; modifying the natural-language output based on a personality profile associated with the persona; and providing the modified natural-language output to a user device.

In some embodiments, the method further comprises one or more of the following: generating, via an analytical model, a data product based on the set of sensor data; generating one or more prompts for a large language model based on one or more of: the data product, user data, plant data, environmental data, a personality profile associated with the persona, or data entered by a user; providing the large language model with the one or more prompts; and receiving a natural-language output from the large language model in response to the one or more prompts.

In some embodiments, the method further comprises: receiving, from the user device, a natural-language user request associated with the plant.

In some embodiments, the natural-language user request is received in one or more of audio, text, and graphical format.

In some embodiments, generating the natural-language output comprises: converting the natural-language user request into machine-language instructions; based on the machine-language instructions, inputting a selection from the set of sensor data into the analytical model; receiving a machine-language output from the analytical model; and converting the machine-language output into the natural-language output.

In some embodiments, the natural-language output is generated based on both the natural-language user request and the set of sensor data.

In some embodiments, the modified natural-language output comprises a response to the natural-language user request according to the personal profile associated with the plant.

In some embodiments, the modified natural-language output comprises a request for one or more responses from the user device.

In some embodiments, the set of sensor data comprises one or more sensor measurements that deviate from one or more predicted measurements generated by the analytical model.

In some embodiments, the request for one or more responses comprises information pertaining to the deviation of the one or more sensor measurements from the one or more predicted measurements.

In some embodiments, the personality profile associated with the plant is configured to be modified based on the deviation of the one or more sensor measurements from the one or more predicted measurements.

In some embodiments, the request for one or more responses comprises a request for feedback pertaining to the analytical model.

In some embodiments, the method further comprises: receiving one or more responses from the user device based on the request.

In some embodiments, the analytical model is modified based on the one or more responses received from the user device.

In some embodiments, the set of sensor data is obtained from one or more sensors associated with the plant.

In some embodiments, the one or more sensors comprise: a) one or more fasteners configured to be positioned in or around a part of the associated plant; b) two or more components selected from the group consisting of: a dendrometer, an accelerometer, an air temperature sensor, a humidity sensor, and a light sensor; c) a processor; and d) a power supply.

In some embodiments, the one or more sensors comprise a dendrometer and an accelerometer.

In some embodiments, the one or more sensors are configured to be positioned in or around the plant.

In some embodiments, the set of sensor data comprises current sensor measurements associated with the plant.

In some embodiments, the set of sensor data comprises historical sensor measurements associated with the plant.

In some embodiments, the set of sensor data comprises data on one or more of air temperature, humidity, light, hydration, plant movement, and plant dimensions.

In some embodiments, the set of sensor data is processed by one or more of an analytical model, a machine-learning algorithm, and an artificial intelligence program.

In some embodiments, the analytical model comprises one or more of a linear model, a temporal fusion transformer, a neural network, a heuristic model, and a decision tree.

In some embodiments, the analytical model is trained on one or more of sensor measurements, plant data, and environmental data.

In some embodiments, modifying the natural-language output comprises: inputting the natural-language output into a large language model; instructing the large language model to modify the natural-language output based on the personality profile; and receiving the modified natural-language output from the large language model.

In some embodiments, the large language model is trained on plant data.

In some embodiments, the large language model is further modified based on the set of sensor data.

In some embodiments, generating the natural-language output comprises: inputting a selection from the set of sensor data into an analytical model; receiving a machine-language output from the analytical model; and converting the machine-language output into the natural-language output.

In some embodiments, the method further comprises modifying the natural-language output based on a user profile associated with the user.

In some embodiments, the modified natural-language output is provided to the user device in one or more of audio, text, and graphical format.

In some embodiments, the modified natural-language output comprises a suggested intervention for the plant.

In some embodiments, the modified natural-language output comprises information pertaining to plant health.

In some embodiments, the modified natural-language output comprises a visual presentation of sensor measurement(s), plant data, or environmental data.

In some embodiments, the personality profile associated with the plant comprises one or more instructions configured to refine the large language model.

In some embodiments, the personality profile associated with the plant is configured to be modified based on the growth rate or transpiration rate of the plant.

In some embodiments, the method further comprises: providing instructions for displaying a graphical representation of the plant at the user device.

In some embodiments, the graphical representation of the plant is based at least in part on a personality profile associated with the plant.

In some embodiments, the graphical representation of the plant is modified based at least in part on sensor data associated with the plant.

In some embodiments, the persona is associated with multiple plants.

In some embodiments, the one or more processors are configured to receive one or more of user data, plant data, and environmental data.

In some embodiments, the one or more processors comprise a moderator program configured to modify the natural-language output based on the personality profile.

In some embodiments, the plant comprises a tree or woody plant.

In some embodiments, the plant is a citrus, olive, nut, cacao, oak, pine, redwood, or maple tree.

In some embodiments, the plant is a vine.

In some embodiments, the vine is a grape vine.

According to some embodiments, provided herein is a system for providing a natural-language interface for conversing with a persona associated with a plant, comprising one or more processors configured to: receive a set of sensor data associated with the plant; generate, via an analytical model, a natural-language output based on the set of sensor data; modify the natural-language output based on a personality profile associated with the persona; and provide the modified natural-language output to a user device.

In some embodiments, the one or more processors are configured to perform any of the methods described herein.

In some embodiments, the system further comprises an application on the user device.

In some embodiments, the system further comprises one or more sensors configured to be positioned in or around the plant.

In some embodiments, to generate the natural-language output, the one or more processors are configured to: input a selection from the set of sensor data into an analytical model; receive a machine-language output from the analytical model; and convert the machine-language output into the natural-language output.

In some embodiments, the system is further configured to receive, from the user device, a natural-language user request associated with the plant.

In some embodiments, to generate the natural-language output, the one or more processors are further configured to: convert the natural-language user request into machine-language instructions; and based on the machine-language instructions, input a selection from the set of sensor data into the analytical model.

In some embodiments, to modify the natural-language output, the one or more processors are configured to: input the natural-language output into a large language model; instruct the large language model to modify the natural-language output based on the personality profile; and receive the modified natural-language output from the large language model.

In some embodiments, the one or more processors comprise a moderator program configured to modify the natural-language output based on the personality profile.

According to some embodiments, provided herein is a non-transitory computer-readable storage medium storing instructions configured to be executed by one or more processors of a system for providing a natural-language interface for conversing with as sensor associated with a plant, wherein executing the instructions causes the system to: receive a set of sensor data associated with the plant; generate, via an analytical model, a natural-language output based on the set of sensor data; modify the natural-language output based on a personality profile associated with the persona; and provide the modified natural-language output to a user device.

In some embodiments, executing the instructions causes the system to perform any of the methods described herein.

In some embodiments, provided herein are methods for providing a natural-language interface for conversing with a persona associated with a device, comprising, at one or more processors: receiving a set of data associated with the device; generating, via an analytical model, a natural-language output based on the set of data; modifying the natural-language output based on a personality profile associated with the persona; and providing the modified natural-language output to a user device. In some embodiments, the set of data associated with the device is related to one or more function(s) of the device and/or one or more measurement(s) obtained via the device.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### DESCRIPTION OF THE FIGURES

The present application can be understood by reference to the following description taken in conjunction with the accompanying figures.

FIG. 1 depicts a system diagram for communicating with plant-based sensors in a natural-language format via artificial intelligence, in accordance with some embodiments.

FIG. 2 depicts a system-initiated method flowchart for providing an output (e.g., a notification about plant health) to a user device in a natural-language format, in accordance with some embodiments.

FIG. 3 depicts a system-initiated method flowchart for modifying an analytical model based on receiving, from a user device in a natural-language format, information regarding an unexpected sensor measurement, in accordance with some embodiments.

FIG. 4 depicts a system-initiated method flowchart for modifying an analytical model based on receiving feedback from a user device in a natural-language format, in accordance with some embodiments.

FIG. 5 depicts a user-initiated method flowchart for requesting an output (e.g., information about plant health) from a user device in a natural-language format, in accordance with some embodiments.

FIG. 6 depicts an exemplary computing device or system, in accordance with some embodiments.

FIG. 7 depicts an exemplary interaction between a user and one or more plant-based sensors, in accordance with some embodiments.

FIG. 8 depicts an exemplary graphical user interface of a mobile application for communicating with plant-based sensors, in accordance with some embodiments.

FIGS. 9A and 9B depict exemplary graphs of dendrometer measurements associated with plant health, in accordance with some embodiments.

### DETAILED DESCRIPTION

The following description sets forth exemplary systems, methods, parameters, and the like. It should be recognized, however, that such description is not intended as a limitation on the scope of the present disclosure but is instead provided as a description of exemplary embodiments.

Described herein are systems and methods for monitoring plant health by communicating with plant-associated persona(s) via artificial intelligence. These systems and methods can communicate information about the health of plants in a natural-language format by using plant-based sensors for collecting data on plant health, processors and analytical models for processing the data on plant health, large language models for converting the data on plant health into a persona-guided, natural-language format, and user devices for receiving the data on plant health in the natural-language format. Also described herein are computer-program products (tangibly embodied in a non-transitory machine-readable storage medium) which include instructions associated with the systems and methods described herein.

The systems and methods described herein provide multiple technical advantages over existing plant health monitoring techniques, such as enabling non-expert users to understand relevant information about the health of their plants and to make educated decisions regarding the caretaking of their plants. Any user, regardless of his or her experience with plant health monitoring, can access these systems and methods through an application on a user device. This has the additional benefit of enabling the user to interact with plant-based sensors remotely, without requiring physical proximity to the plant. Furthermore, because the processing of plant health data is performed by a trained analytical model and does not rely on the user's expertise, the systems and methods described herein can have a higher degree of confidence than existing plant health monitoring techniques. In some instances, analytical model(s) about plant health and various plant parameter can be improved by the systems and methods of the present disclosure, e.g., by soliciting user input/feedback and updating the analytical model(s) in response, particularly when measurement(s) associated with the plant (e.g., from an associated sensor of the present disclosure) deviate from the analytical model(s).

Systems for Communicating with Plant-Based Sensors Via Artificial Intelligence

According to some embodiments, large language model (LLM) artificial intelligence (AI) software and software services, such as OpenAI's ChatGPT and GPT-4, can be pre-trained on vast amounts of information, including plant care, gardening, horticulture and agriculture information. These LLMs can accurately answer questions about tree care, provided they are prompted with appropriate information and guidance. Effective prompts can include, but are not limited to, background information about a tree, its location, current situation, history, and weather conditions. The prompts can also guide the LLMs to adopt colorful personalities and interact in surprising, playful, and/or humorous ways to answer questions and deliver information. When suitably prompted, these artificial intelligence models can be effective at humanizing the presentation of technical information.

According to some embodiments, a system described herein can exchange, with both expert and non-expert users alike, accurate and timely information, guidance, notification, feedback, or answers regarding plant health and caretaking. Users can interact with the systems and devices of the present disclosure via a natural-language format conversational interface supported by a LLM, as described herein. Plant-based (i.e., plant-mounted) sensors connected to a distributed Internet of Things (IoT) network can generate sensor data used by various components of the system. When viewed in the context of weather, plant imagery, and plant metadata, such as plant type, location, soil conditions, or irrigation configuration, sensor data generated by these plant-based sensors can reveal important information about plant health. For example, over periods of time ranging from hours to years, sensor data can provide users with insights related to plant hydration, nutrition, or growth, thereby enabling users to optimize water or fertilizer application.

According to some embodiments, sensor data can be processed by a cloud-based analytical model to produce insights, observations, classifications, or characterizations for each plant. Additionally, user data, environmental data, plant data, and other types of data can be processed by the analytical model to provide further insights on the sensor data. The analytical model can be configured to generate notifications or alerts according to users' preferences. For example, based on the processing of the analytical model, the system described herein can produce a mobile phone notification on a user's phone, informing the user that a plant may require water. The analytical model can also be configured to provide processed sensor data to a “conversation moderator” program running on one or more processors of the system. The “conversation moderator” can be configured to provide relevant information, such as conversational constraints, background information, or a personality profile associated with the plant, to a LLM. For example, a user can interact with the system in a natural-language format via a web-based chat interface to ask questions about plant hydration, and subsequently, the system can provide the user with a natural-language answer that has been processed by the analytical model, generated by the LLM, and moderated by the “conversation moderator.” Throughout the user's interaction with the system and any subsequent interactions, the “conversation moderator” can support follow-up questions and maintain a consistent linguistic tone or personality profile associated with each plant.

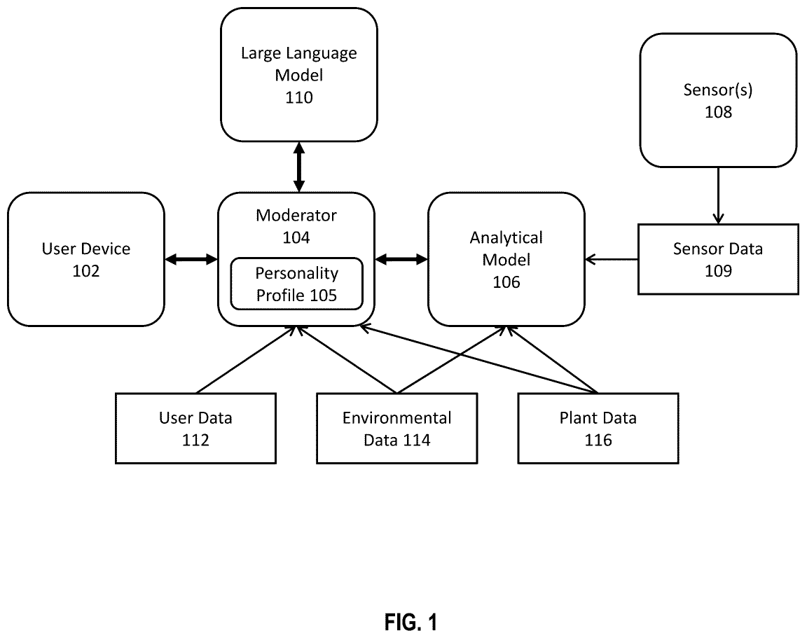

FIG. 1 depicts a simplified block diagram of a system 100 for communicating with plant-associated persona(s) in a natural-language format via artificial intelligence, in accordance with some embodiments. In the exemplary embodiment, the system 100 can include a user device 102, processors 104, a personality profile 105, an analytical model 106, sensor(s) 108, sensor data 109, a large language model 110, user data 112, environmental data 114, and plant data 116. The system 100 can be operated in a variety of methods, including, but not limited to, method 200, method 300, method 400, method 500, or any combinations thereof.

In some embodiments, the user device 102 can comprise an electronic device, including, but not limited to, a phone, tablet, laptop, or computer. The user device 102 can have any suitable form factor. The user device 102 can include one or more processors; one or more user interfaces; one or more input/output components such as a display screen, keyboard, mouse, microphone, or speaker; one or more storage or memory components; one or more network interfaces for communicating with external servers or devices; and one or more software components. A user can interact with system 100 through user device 102 via an application, program, or other software component on the user device 102. For example, when interacting with system 100 via a phone application, the user can speak into a microphone to input a voice-based message, or type on a digital keyboard of a display screen to input a text-based message into the phone application. Additionally, the user device 102 can send messages to the user through the phone application, which can output a voice-based message through a speaker or a text-based message through the display screen.

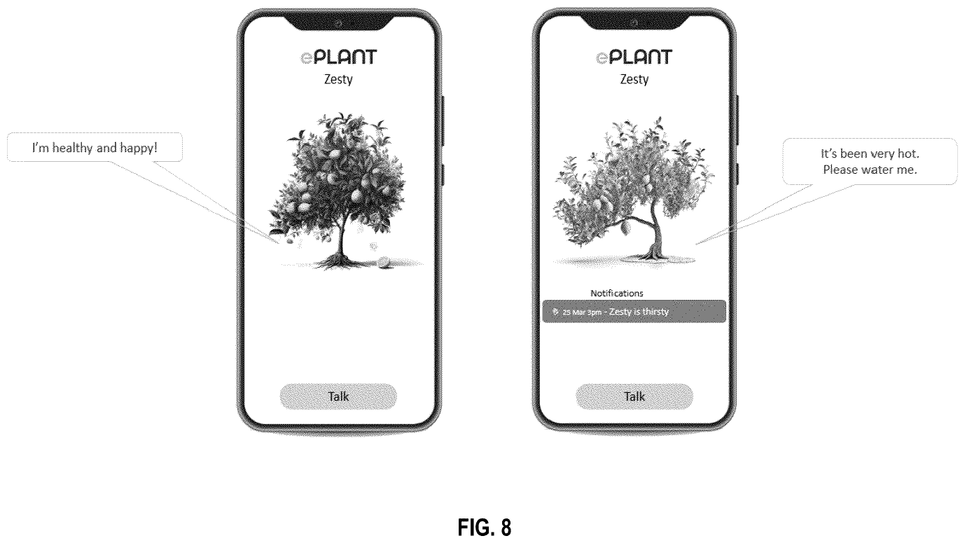

In some embodiments, the moderator 104 can comprise a program configured to moderate the conversation between a user and the system 100. Specifically, the moderator 104 can comprise, on one or more processors, a program configured to modify a natural-language output, such as a message to be displayed on the user device 102. The moderator 104 can format any information to be provided to the large language model 110, including, but not limited to, the sensor data 109, the user data 112, the environmental data 114, the plant data 116. Specifically, this information can include one or more of language preference (e.g., English or Spanish, etc.), conversational tone, insights from the analytical model 106, weather forecast data, and the timing of user-initiated events (e.g., irrigation). The modification to conversational tone can be based on a personality profile 105 associated with the plant. The personality profile 105 can be part of the moderator 104, stored on the same one or more processors as the moderator 104, or stored externally from the moderator 104 (e.g., on the user device 102), depending on the configuration of the system 100. The personality profile 105 can comprise one or more instructions for refining the large language model 110 or its outputs. Additionally, the personality profile 105 can be modified based on factors such as the growth rate or the transpiration rate of the plant. For example, when generating responses from the perspective of a lemon tree that appears to be under severe water stress, the large language model 110 can receive instructions from the personality profile 105 to adopt a panicked or annoyed affect in the conversational responses. Conversely, when the lemon tree is rapidly growing, the large language model 110 can receive instructions to adopt a happy and excited tone. In another example, the personality profile 105 can be associated with a plant caretaker, such as a garden gnome who is taking care of multiple plants. In some embodiments, a graphical representation of the plant can be displayed, e.g., at user device 102. For example, the moderator 104 can instruct user device 102 to display a graphical representation or avatar associated with the plant. In some embodiments, the graphical representation or avatar is based at least in part on the personality profile 105 and/or one or more plant characteristics (e.g., youthful features for a younger plant, representation of fruit for a fruit tree or vine, etc.). In some embodiments, the graphical representation or avatar is based at least in part on sensor data and/or an analytical model associated with the plant (e.g., a drier appearance if the sensor data and/or analytical model indicate the plant is undergoing water stress, etc.). For example, as shown in FIG. 8, the avatar for the lemon tree (“Zesty”) in the left panel is green and healthy when sensor data indicate the plant is healthy, but less vibrant and shriveled in appearance in the right panel when sensor data indicate the plant needs water. As such, the graphical representation or avatar can convey complex concepts or plant needs in a readily understandable way via updates to its appearance.

In an exemplary embodiment, the moderator 104 can be configured to provide relevant information, such as conversational constraints, background information, or a personality profile 105 associated with the plant, to the large language model 110. The moderator 104 may provide large headers, expanded prompts, or system definition text files to shape the response of the large language model 110 in terms of content, tone, personality, or language style. For example, a user can interact with the system 100 in a natural-language format via a web-based chat interface (i.e., user device 102) to ask questions about plant hydration, and subsequently, the system 100 can provide the user with a natural-language answer that has been processed by the analytical model 106, generated by the large language model 110, and moderated by the moderator 104. Throughout the user's interaction with the system 100 and any subsequent interactions, the moderator 104 can support follow-up questions and maintain a consistent linguistic tone (i.e., personality profile 105) associated with each plant.

In some embodiments, the analytical model 106 can comprise a trained analytical or predictive model, machine-learning algorithm, or other artificial intelligence program, including, but not limited to, a linear model, a temporal fusion transformer, a neural network, or a decision tree. The analytical model 106 may be used to generate insights, such as predictions, optimizations, or other analyses, regarding plant health and caretaking. Training data for the analytical model 106 can include the sensor data 109 and the environmental data 114 for a specific plant, as well as datasets from a large group of plants and environments. Furthermore, the analytical model 106 can be re-trained or modified as needed based on additional datasets, user responses, or user feedback (refer to method 300 and method 400, described herein, for examples). The analytical model 106 can receive and process one or more of the sensor data 109, the user data 112, the environmental data 114, and the plant data 116. The user data 112, the environmental data 114, or the plant data 116 can be processed by the analytical model 106 to provide further insights on the sensor data 109. “Processing” by the analytical model 106 can involve one or both of algorithmic processing or processing by machine learning techniques. The analytical model 106 can be configured to provide the processed sensor data 109 to a “conversation moderator” program of the moderator 104. Additionally, the analytical model 106 can be configured to generate notifications or alerts regarding the health of the plant based on the processed sensor data 109. For example, the analytical model 10 can process temperature data (i.e., sensor data 109) from a thermometer (i.e., sensor(s) 108) in the context of data from a weather forecast (i.e., environmental data 114) to produce a frost alert.

In an exemplary embodiment, the analytical model 106 can comprise a plurality of cloud-based software components which process the above data to produce insights, observations, classifications, or characterizations for a plant. For example, the analytical model 106 can process tree water deficit (TWD) data (i.e., sensor data 109) from a dendrometer (i.e., sensor(s) 108) in the context of light intensity, air temperature, and humidity (i.e., environmental data 114) to produce an estimate of a plant's water response. Based on the processing of the analytical model 106, the system 100 can produce a mobile phone notification on a user's phone (i.e., user device 102), informing the user that a plant may require water.

In an exemplary embodiment, by processing sensor data 109, the analytical model 106 can generate predictions based on historical sensor measurements, then compare the predictions to current sensor measurements to identify deviations. Furthermore, plant data 116 from plants of similar species, types, locations, and conditions can be used to refine the predictions generated by, as well as the deviations identified by, the analytical model 106. The personality profile 105 can be modified based on the deviations between the predictions and the current sensor measurements, thereby affecting the messages produced by the system 100. For example, upon the analytical model 106 detecting a deviation, the system 100 can produce a natural-language notification on a user's phone (i.e., user device 102) from the perspective of the plant, asking the user a question such as “Hey, did you water me recently? I'm feeling hydrated!” In another example, the analytical model 106 can request feedback from the user device 102 to modify and improve the processing of data without first detecting a deviation. To accomplish this, the system 100 can ask a question, such as “Do you think the plant is well-watered right now?” The user can respond “Yes” to the question via the user device 102, and based on the response, the analytical model 106 can modify its future insights accordingly.

In some embodiments, the sensor(s) 108 can comprise one or more plant-based sensors for obtaining data associated with a plant. The sensor(s) 108 can comprise components including, but not limited to, dendrometers, accelerometers, air temperature sensors, humidity sensors, light sensors, or sap flow sensors. In an exemplary embodiment, the sensors 108 can comprise two or more components selected from the sensors above (e.g., a dendrometer and an accelerometer), one or more fasteners configured to be positioned in or around a plant part (such that the sensor is positioned in or around the plant part), a processor, and a power supply. In some embodiments, the sensor(s) 108 are configured to provide real-time measurements of the plant or plant part of the present disclosure. Any of the plant sensors and aspects thereof described in International Publication No. WO2023034380 are contemplated for use in the present disclosure.

In some embodiments, the sensor(s) 108 can comprise a printed circuit board (PCB). In some embodiments, one or more components is/are affixed to the PCB. In some embodiments, all of the components are affixed to the PCB. In some embodiments, the PCB comprises an epoxy-fiberglass composite material. In some embodiments, the power supply comprises a battery. In some embodiments, the battery is a coin cell battery. In some embodiments, the battery is affixed to the PCB. In some embodiments, the power supply comprises a solar panel. In some embodiments, the power supply comprises an integrated solar panel, hybrid capacitor, and lithium battery. In some embodiments, the solar panel is affixed to the PCB. In some embodiments, the sensor further comprises a housing, e.g., that encloses at least the processor and power supply. In some embodiments, the housing is or comprises plastic, e.g., molded plastic. In some embodiments, the processor and magnetometer are enclosed in a sealed, overmolded housing comprising an O-ring. In some embodiments, the overmolded housing comprises a removable lid covering the battery. In some embodiments, the housing is a single piece of overmolded plastic that lacks a seal, junction, or fastener.

In some embodiments, the sensor(s) 108 can comprise a dendrometer. In some embodiments, the dendrometer comprises: a plunger having a cap and a shaft, wherein the cap is configured to be positioned against a plant part, and wherein the plunger is configured to move laterally in proportion to a change in plant size when the cap is positioned against the plant part; a magnet attached to or within the shaft, wherein the magnet is configured to move laterally in association with the plunger; and a magnetometer configured to detect position of the magnet. In some embodiments, the magnetometer is configured to detect position of the magnet along multiple axes, a radial axis, or a single plane. In some embodiments, the magnetometer is configured to detect position of the magnet at micron-scale resolution. In some embodiments, the magnetometer is configured to detect position of the magnet along multiple axes, e.g., along a radial axis. In some embodiments, the magnetometer is configured to detect position of the magnet using a ratiometric measurement.

In some embodiments, the sensor(s) 108 can comprise an accelerometer. In some embodiments, the accelerometer is a 3-axis accelerometer. In some embodiments, the processor comprises a PCB, and the accelerometer is affixed to the PCB. In some embodiments, the sensor comprises a light sensor. In some embodiments, the processor comprises a PCB, and the light sensor is affixed to the PCB. In some embodiments, the sensor comprises a humidity sensor. In some embodiments, the processor comprises a PCB, and the humidity sensor is affixed to the PCB. In some embodiments, the sensor comprises an air temperature sensor. In some embodiments, the processor comprises a PCB, and the air temperature sensor is affixed to the PCB.

In some embodiments, the sensor(s) 108 can further comprise a transmitter or transceiver. In some embodiments, the transmitter is a Bluetooth radio or transceiver, e.g., a Bluetooth Low Energy (BLE) radio or transceiver. In some embodiments, the transmitter is a Long Range (LoRa) transceiver. In some embodiments, the transmitter is a Near Field Communication (NFC) transceiver. In some embodiments, the transmitter is affixed to the PCB. In some embodiments, the sensor(s) 108 are connected to the moderator 104 and/or user device 102 via Bluetooth low energy (BLE), Long Range (LoRa), or a combination thereof. In some embodiments, the transmitter includes is configured to transmit sensory data wirelessly (e.g., Bluetooth, WiFi, or 900 MHz transmitter) to a mobile device or server. Other possible wireless networks include Narrowband Internet of Things (IoT), LTE-M, and satellite-based networks such as Myriota or Swarm. In some embodiments, the transmitter is a radio. In some embodiments, the transmitter is a transceiver (e.g., Bluetooth transceiver, WiFi transceiver, etc.). In some embodiments, the transmitter is a Long Range (LoRa) transceiver or Near Field Communication (NFC) transceiver. In some embodiments, the transmitter uses Lora radio data transmission system or the LoraWAN network protocol. Advantageously, this provides low-power, long-range transmission. In some embodiments, the transmitter uses a frequency band of about 900 MHz. In some embodiments, the sensor comprises a chip antenna, e.g., the Ignion NN2-2204. In some embodiments, the sensor comprises a split dipole antenna, and two wires extend on opposite sides of the sensor. In some embodiments, the transmitter uses a frequency band of about 900 MHz, and the sensor has a ground plane that is approximately 72 mm (quarter wave length for 900 MHz frequency band) or longer to complement an active antenna side that may be a single wire extending in the opposite direction of the ground plane (up, if the solar panel is down from the mount screw). In some embodiments, the ground plane of the device may be shared with the solar panel.

In some embodiments, the one or more fasteners comprises a screw, threaded rod, or nail, and wherein the screw, threaded rod, or nail is configured to be positioned within a plant part and mount the sensor to the plant part. In some embodiments, the one or more fasteners comprises one or more curved arm(s), wherein the curved arm(s) are configured to be positioned around a plant part. In some embodiments, the one or more fasteners comprises two curved arms arranged in a U- or V-shape. In some embodiments, the curved arm(s) are configured to be positioned around the plant part opposite the plunger cap. In some embodiments, the one or more fasteners further comprises an elastic band configured to be wrapped around the sensor and the plant part.

In an exemplary embodiment, the health of a variety of plants may be monitored with one or more sensor(s) 108. The sensor(s) 108 can be used to measure any type of plant including, but not limited to, vegetables (e.g., tomatoes, etc.), trees (e.g., rubber trees, fruit trees, etc.), row crops, ornamental plants, and the like. In some embodiments, the plant can be a crop tree. In some embodiments, the plant can be a citrus, olive, nut, cacao, oak, pine, redwood, “strawberry,” or maple tree. In some embodiments, the plant can be a woody plant. In some embodiments, the plant can be a vine (e.g., a grape vine). In some embodiments, the sensor(s) 108 can be attached to various part(s) of the plant, including without limitation stem, trunk, bole, branch, vine, shoot, cane, or fruit.

In some embodiments, the sensor data 109 can comprise data or metadata associated with or obtained from the sensor(s) 108. The sensor data 109 can include plant measurements such as those associated with dendrometers, accelerometers, humidity, temperature, light intensity, lean direction, lean amount, plant movement (e.g., swaying, shaking, or tilting), sap flow rate, passive electrical signals, impedance, acoustic monitoring (e.g., of sap flow or pests), plant dimensions, or water potential. These plant measurements can be current sensor measurements, as well as historical sensor measurements recorded and aggregated over a period of time. The sensor data 109 can be associated with the plant of the sensor(s) 108, as well as other plants and other sensors. The sensor data 109 can be processed by one or more of an analytical model, a machine-learning algorithm, and an artificial intelligence program, such as the analytical model 106.

Advantageously, collecting data from multiple sensors can be used to compensate measurement of diameter change, indirectly compensate to account for mixed signal from bark that could obscure signal from the living plant layers, calibrate and cross-validate data from multiple sources, and understand the drivers of tree growth and/or daily expansion/contraction. For example, these data can be used to approximate and/or predict vapor pressure deficit (VPD) and thereby predict organism dendrometry response. Data can be sent to a server or mobile device via the antenna, creating a distributed IoT network for data collection. These data are high resolution, real-time, and can be collected in a system (e.g., comprising multiple sensors mounted to multiple plants) in which comparisons between multiple organisms can be done (e.g., comparing growth between organisms in similar states, of comparable species, in comparable geographic regions, in comparable weather conditions, in comparable soil conditions, under comparable care/watering/irrigation regimes, etc.). Using these data, model(s) can be constructed for each organism based on observed dendrometry signal, collected environmental or weather data, etc. to predict future dendrometry, e.g., based on current environmental signals or conditions. Further, variance from the model can help to indicate non-measured factors including soil moisture, pests, disease, toxicity, predation, damage, and so forth.

In some embodiments, the large language model 110 can comprise LLM AI software and software services, such as Google's Bard or OpenAI's ChatGPT and GPT-4, which generate natural-language responses when prompted. The large language model 110 can be pre-trained on vast amounts of information, including plant care, gardening, horticulture and agriculture information. The large language model 110 receives prompts from the moderator 104, which can include instructions to adopt a conversational tone (in line with personality profile 105) while generating responses. When prompted with accurate information, such as information provided by the moderator 104, the large language model 110 can accurately generate responses to questions about, for example, tree care. The generated responses can be received by the moderator 104, which can provide the large language model 110 with additional prompts to refine or modify the generated responses. This back-and-forth interaction between the large language model 110 and the moderator 104 can enable the system 100 to generate natural-language responses in line with personality profile 105. When suitably prompted, the large language model 110 can be effective at humanizing the presentation of technical information related to plants.

In some embodiments, the user data 112 can comprise data or metadata associated with the user of the system 100. The user data 112 can be obtained from user input. The user data 112 can include user-reported events such as irrigation, pruning, and fertilizer application. Additionally, the user data 112 can include data about the user's age, familiarity with plant caretaking, and conversation preferences. For example, if a user prefers to receive messages in a friendly tone with vocabulary appropriate for a middle school audience, this information may be included in the user data 112. The moderator 104 or the analytical model 106 can be configured to receive and process the user data 112.

In some embodiments, the environmental data 114 can comprise data or metadata associated with the environment of the plant, including, but not limited to, weather data. The environmental data 114 can be obtained from local weather stations or from weather data services based on location. The environmental data 114 can include atmospheric measurements such as those associated with temperature, humidity, wind speed, precipitation, or air pressure. These measurements can be used to calculate estimates of vapor pressure deficit (VPD) or evapotranspiration (ET), which can be used to assess or predict the health of the plant. The moderator 104 or the analytical model 106 can be configured to receive and process the environmental data 114.

In some embodiments, the plant data 116 can comprise data or metadata associated with the plant, including, but not limited to, plant species, type, location, age, soil condition, sun exposure/shade, pruning schedule, or irrigation configuration. The plant data 116 can be obtained from user input, a nursery tag, or a pre-existing database of plant information. The moderator 104 or the analytical model 106 can be configured to receive and process the plant data 116.

The systems and methods described herein are not inherently related to any particular computer or other apparatus. Various general-purpose systems may also be used with programs in accordance with the teachings herein, or it may prove convenient to construct a more specialized apparatus to perform the required method steps. The required structure for a variety of these systems will appear from the description above. In addition, the present disclosure is not described with reference to any particular programming language. It will be appreciated that a variety of programming languages may be used to implement the teachings of the present disclosure as described herein.

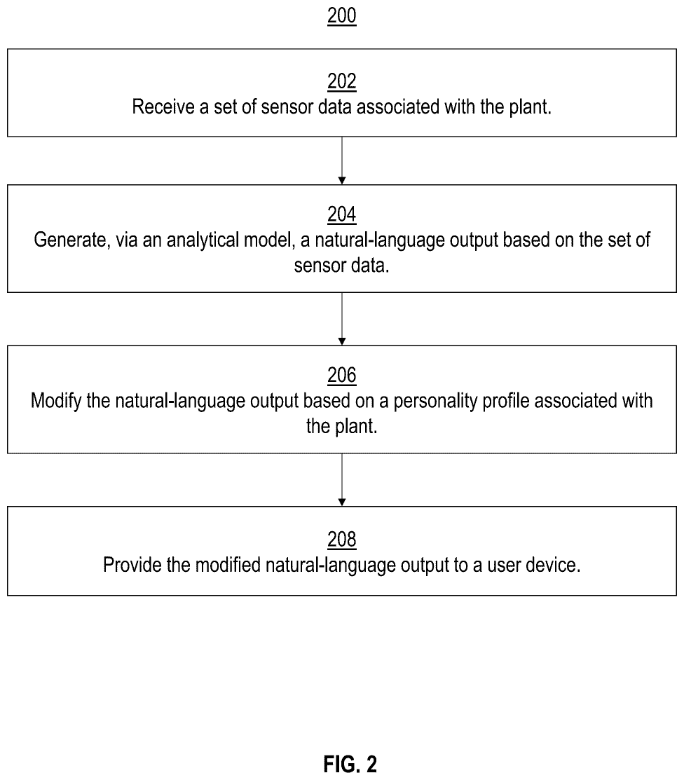

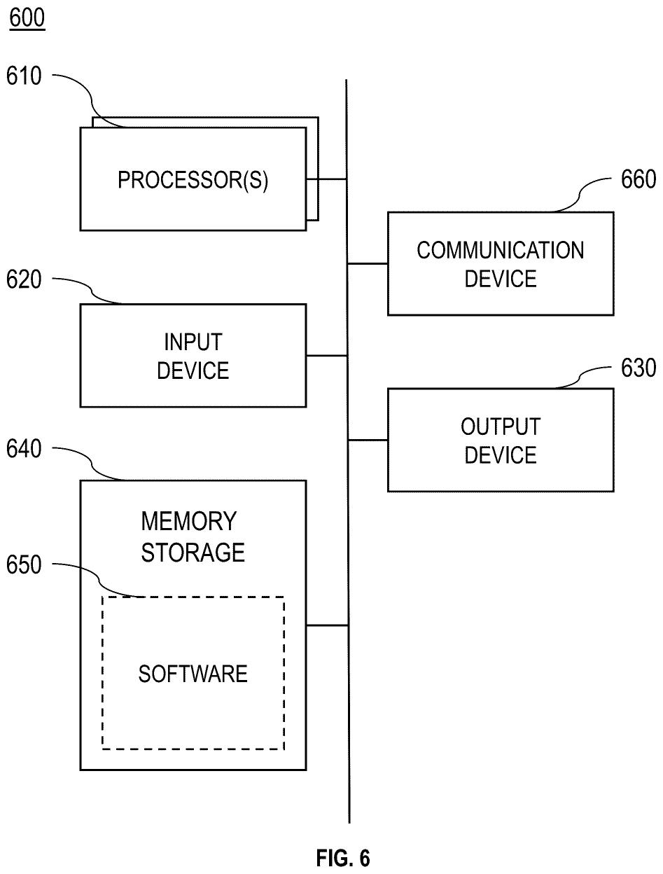

FIG. 6 depicts an exemplary computing device or system, in accordance with some embodiments. Computer 600 can be a component of a system for communicating with plant-associated persona(s) according to the systems and methods described above. In some embodiments, computer 600 is configured to execute each of methods 200, 300, 400, and 500 of FIGS. 2, 3, 4, and 5, respectively.

Computer 600 can be a host computer connected to a network. Computer 600 can be a client computer or a server. As shown in FIG. 6, computer 600 can be any suitable type of microprocessor-based device, such as a personal computer, workstation, server, videogame console, or handheld computing device, such as a phone or tablet. The computer can include, for example, one or more of processor 610, input device 620, output device 630, storage 640, and communication device 660. Input device 620 and output device 630 can generally correspond to those described above and can either be connectable or integrated with the computer.

Input device 620 can be any suitable device that provides input, such as a touch screen or monitor, keyboard, mouse, or voice-recognition device. Output device 630 can be any suitable device that provides output, such as a touch screen, monitor, printer, disk drive, or speaker.

Storage 640 can be any suitable device that provides storage, such as an electrical, magnetic, or optical memory, including a RAM, cache, hard drive, CD-ROM drive, tape drive, or removable storage disk. Communication device 660 can include any suitable device capable of transmitting and receiving signals over a network, such as a network interface chip or card. The components of the computer can be connected in any suitable manner, such as via a physical bus or wirelessly. Storage 640 can be a non-transitory computer-readable storage medium comprising one or more programs, which, when executed by one or more processors, such as processor 610, cause the one or more processors to execute methods described herein, such as each of methods 200, 300, 400, and 500 of FIGS. 2, 3, 4, and 5, respectively.

Software 650, which can be stored in storage 640 and executed by processor 610, can include, for example, the programming that embodies the functionality of the present disclosure (e.g., as embodied in the systems, computers, servers, and/or devices as described above). In some embodiments, software 650 can be implemented and executed on a combination of servers such as application servers and database servers.

Software 650, or part thereof, can also be stored and/or transported within any computer-readable storage medium for use by or in connection with an instruction execution system, apparatus, or device, such as those described above, that can fetch and execute instructions associated with the software from the instruction execution system, apparatus, or device. In the context of this disclosure, a computer-readable storage medium can be any medium, such as storage 640, that can contain or store programming for use by or in connection with an instruction execution system, apparatus, or device.

Software 650 can also be propagated within any transport medium for use by or in connection with an instruction execution system, apparatus, or device, such as those described above, that can fetch and execute instructions associated with the software from the instruction execution system, apparatus, or device. In the context of this disclosure, a transport medium can be any medium that can communicate, propagate, or transport programming for use by or in connection with an instruction execution system, apparatus, or device. The transport-readable medium can include, but is not limited to, an electronic, magnetic, optical, electromagnetic, or infrared wired or wireless propagation medium.

Computer 600 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Computer 600 can implement any operating system suitable for operating on the network. Software 650 can be written in any suitable programming language, such as C, C++, Java, or Python. In various embodiments, application software embodying the functionality of the present disclosure can be deployed in different configurations, such as in a client/server arrangement or through a Web browser as a Web-based application or Web service, for example.

In some embodiments, a non-transitory computer readable storage medium stores one or more programs configured to be executed by one or more processors of a computing device, the one or more programs including instructions for implementing any of the steps described or claimed herein. The present disclosure also relates to a device for performing the operations herein. This device may be specially constructed for the required purposes, or it may include a general purpose computer selectively activated or reconfigured by a computer program stored in the computer. Such a computer program may be stored in a non-transitory, computer readable storage medium, such as, but not limited to, any type of disk, including floppy disks, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each coupled to a computer system bus. Furthermore, the computers referenced in this disclosure may include a single processor or may be architectures employing multiple processor designs for increased computing capability.

Methods for Communicating with Plant-Associated Persona(s) Via Artificial Intelligence

According to some embodiments, methods described herein can enable expert and non-expert users alike to interact with a system to exchange accurate and timely information, guidance, notification, feedback, or answers regarding plant health and caretaking. The methods described herein can be roughly divided into two modes: (1) a “Talking Tree” mode for communicating with users to share insights related to plant health, and (2) a “Learning Tree” mode for receiving user responses or feedback to refine the analytical model that generates the insights for the “Talking Tree” mode.

### “Talking Tree” Mode

According to some embodiments, “Talking Tree” mode can include methods of communicating with users to share insights related to plant health. Method 200 and method 500 are examples of “Talking Tree” mode. Method 200 can comprise a system-initiated method for providing an output (i.e., insights related to plant health) to a user device in a natural-language format. Likewise, method 500 can comprise a user-initiated method for requesting an output (i.e., insights related to plant health) in a natural-language format.

FIG. 2 depicts a simplified flowchart of a system-initiated method 200 for providing an output (e.g., a notification about plant health) to a user device in a natural-language format, in accordance with some embodiments. At block 202, a set of sensor data associated with a plant can be received. At block 204, a natural-language output based on the set of sensor data can be generated via an analytical model. At block 206, the natural-language output can be modified based on a personality profile associated with the plant. At block 208, the modified natural-language output can be provided to the user device. This modified natural-language output can be a notification about plant health (e.g., a notification that a tree requires water). Method 200 can be initiated by the system based on the received sensor data. User input is not needed to initiate method 200.

In some embodiments, method 200 can be performed, for example, using one or more electronic devices implementing a software platform. In an exemplary example, method 200 can be performed by system 100. In some examples, method 200 can be performed using a client-server system, and the blocks of method 200 can be divided up in any manner between a server and one or more client devices (i.e., user devices). In other examples, method 200 can be performed using only a client device or only multiple client devices. Thus, while portions of method 200 are described herein as being performed by particular devices of a client-server system, it will be appreciated that method 200 is not so limited. In method 200, some blocks can, optionally, be combined, the order of some blocks can, optionally, be changed, and some blocks can, optionally, be omitted. In some examples, additional steps can be performed in combination with the method 200. Accordingly, the operations as illustrated (and described in greater detail below) are exemplary by nature and, as such, should not be viewed as limiting.

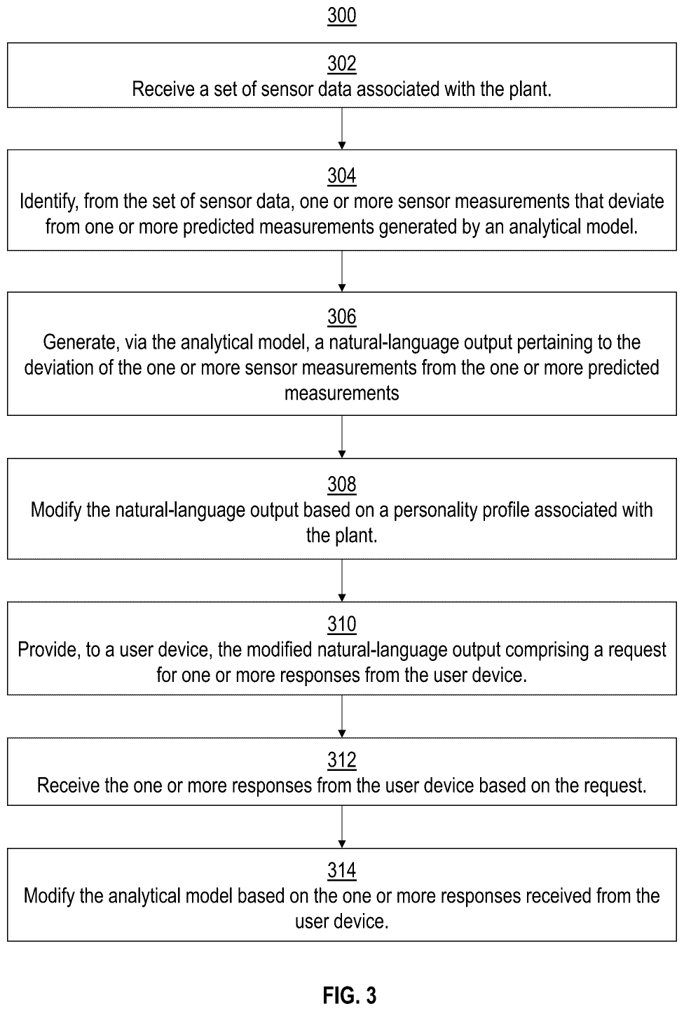

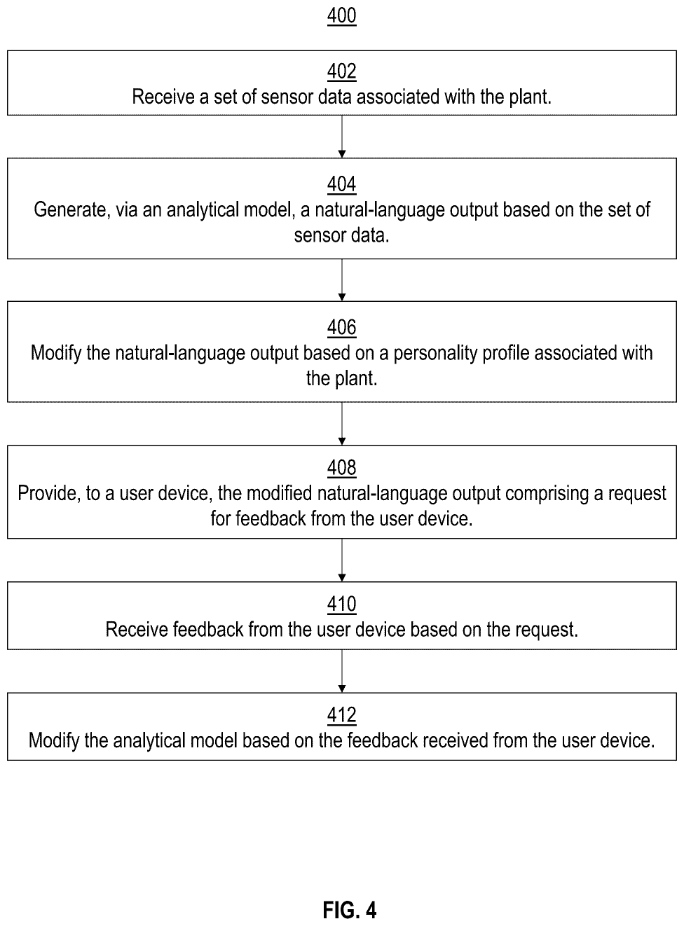

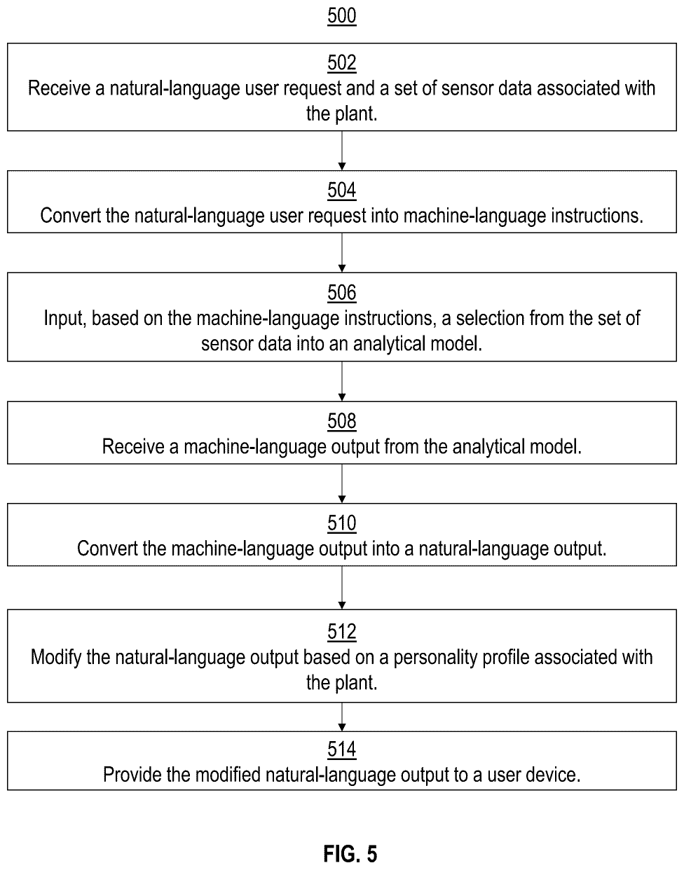

In some embodiments, at block 202, a set of sensor data associated with the plant is received. The sensor data can include any features associated with sensor data 109 from FIG. 1. For example, the processors can receive dendrometer measurements that include data on a plant's size across a period of time. Block 202 can include any features of block 302 of FIG. 3, block 402 of FIG. 4, and/or block 502 of FIG. 5, and vice versa.

In some embodiments, at block 204, a natural-language output based on the set of sensor data can be generated via an analytical model. Generating the natural-language output can comprise inputting a selection from the set of sensor data into an analytical model, receiving a machine-language output from the analytical model, and converting the machine-language output into the natural-language output. For example, the processors can send a selection of dendrometer measurements from the past week to the analytical model, and then the analytical model can generate insights on the growth of the plant over the past week. These insights can be processed by a large language model to be converted into a natural-language message about the plant's growth. Block 204 can include any features of block 306 of FIG. 3, block 404 of FIG. 4, and/or blocks 504 to 510 of FIG. 5, and vice versa.

In some embodiments, at block 204, instead of a natural-language output, a data product based on the set of sensor data can be generated via an analytical model. For example, the data product can be sensor data that has been analyzed to produce outputs compatible with a large language model. The data product can be used alone or in conjunction with one or more of user data, plant data, environmental data, a personality profile associated with the persona, and data entered by a user to generate one or more prompts for the large language model. The one or more prompts can then be provided to the large language model to generate a natural-language output in response to the one or more prompts.

In some embodiments, at block 206, the natural-language output can be modified based on a personality profile associated with the plant. The personality profile can include any features associated with personality profile 105 of FIG. 1. In some embodiments, the moderator program of the processors can instruct a large language model to modify a natural-language message according to the personality profile. For example, if a plant is associated with a cheerful personality profile, a message about the plant's growth can be modified to say “Yay! I'm growing quickly!” Additionally, the natural-language output can be modified based on data associated with the user, such as a user profile, previous user responses, or user metadata. For example, a user can specify a language preference (e.g., English or Spanish, etc.) or age-appropriate vocabulary level (e.g., for an elementary school vocabulary level), which modifies the natural-language output accordingly. In another example, location data about the user can be used to modify the natural-language output to suggest different products or plant care services relevant to the user's location. In another example, the natural-language output format can be modified according to the user's needs, such as providing audio-format outputs for visually impaired users, text- or graphical-format outputs for hearing impaired users, or graphical-format outputs for users who are young children. Block 206 can include any features of block 308 of FIG. 3, block 406 of FIG. 4, and/or block 512 of FIG. 5, and vice versa.

In some embodiments, at block 208, the modified natural-language output can be provided to the user device. The modified natural-language output can be provided in one or more of audio, text, and graphical format. In some embodiments, the modified natural-language output can comprise a suggested intervention for the plant. For example, the output can be a message informing the user that the plant needs more or less water, a specific type or amount of fertilizer, pruning or trimming, more or less sun exposure, or the application of a fungicide or pest treatment. In some embodiments, the modified natural-language output can comprise information pertaining to plant health. For example, the output can be a message informing the user that a plant is very healthy and happy with the care it has been receiving. In some embodiments, the modified natural-language output can comprise a visual presentation of sensor measurements, plant data, or environmental data. For example, the output may be a graph showing that a plant's water response is weaker during hotter days and stronger during cooler nights. Block 208 can include any features of block 310 of FIG. 3, block 408 of FIG. 4, and/or block 514 of FIG. 5, and vice versa.

FIG. 5 depicts a simplified flowchart of a user-initiated method 500 for requesting an output (e.g., information about plant health) from a user device in a natural-language format, in accordance with some embodiments. At block 502, a natural-language user request and a set of sensor data associated with a plant can be received. At block 504, the natural-language user request can be converted into machine-language instructions for an analytical model. At block 506, a selection from the set of sensor data can be inputted into the analytical model based on the machine-language instructions. At block 508, a machine-language output can be received from the analytical model. At block 510, the machine-language output can be converted into a natural-language output. At block 512, the natural-language output can be modified based on a personality profile associated with the plant. At block 514, the modified natural-language output can be provided to the user device. This modified natural-language output can be user-requested information about plant health (e.g., in response to a user asking if a tree is thirsty, the tree can respond with information about its water retention). Method 500 can be initiated by the user based on the user's prompts or requests.

In some embodiments, method 500 can be performed, for example, using one or more electronic devices implementing a software platform. In an exemplary example, method 500 can be performed by system 100. In some examples, method 500 can be performed using a client-server system, and the blocks of method 500 can be divided up in any manner between a server and one or more client devices (i.e., user devices). In other examples, method 500 can be performed using only a client device or only multiple client devices. Thus, while portions of method 500 are described herein as being performed by particular devices of a client-server system, it will be appreciated that method 500 is not so limited. In method 500, some blocks can, optionally, be combined, the order of some blocks can, optionally, be changed, and some blocks can, optionally, be omitted. In some examples, additional steps can be performed in combination with the method 500. Accordingly, the operations as illustrated (and described in greater detail below) are exemplary by nature and, as such, should not be viewed as limiting.

In some embodiments, at block 502, a natural-language user request and a set of sensor data associated with the plant is received. The natural-language user request can be received in one or more of audio, text, and graphical format. The natural-language user request is associated with the plant and can include a prompt, question, or other message from a user interacting with the system. For example, the natural-language user request may be a question directed to the plant-associated persona(s), such as “Do you need water?” The sensor data can include any features associated with sensor data 109 from FIG. 1. Block 502 can include any features of block 202 of FIG. 2, block 302 of FIG. 3, and/or block 402 of FIG. 4, and vice versa.

In some embodiments, blocks 504 to 510 can comprise generating, via an analytical model, a natural-language output based on the natural-language user request and the set of sensor data. Blocks 504 to 510 can include any features of block 204 of FIG. 2, block 306 of FIG. 3, and/or block 404 of FIG. 4, and vice versa.

In some embodiments, at block 504, the natural-language user request can be converted into machine-language instructions. These machine-language instructions can include one or more inputs compatible with the analytical model, including, but not limited to, electronic signals, binary signals, code such as a low-level programming code, or assembly language. For example, upon receiving a natural-language user request comprising the question “Do you need water?”, the processors can convert the request into a machine-language instruction for identifying and processing sensor data related to the plant's water response.

In some embodiments, at block 506, a selection from the set of sensor data can be inputted into the analytical model based on the machine-language instructions. For example, upon receiving a machine language-instruction for identifying and processing sensor data related to the plant's water response, the processors can input the relevant sensor data into the analytical model to yield insights about the plant's water response.

In some embodiments, at block 508, a machine-language output can be received from the analytical model. For example, insights about the plant's water response that have been generated by the analytical model may be received by the processors in a machine-language format.

In some embodiments, at block 510, the machine-language output can be converted into a natural-language output. The processors may convert the insights generated by the analytical model from a machine-language format to a natural-language format that responds to the user request. For example, the natural-language user request may be a question directed to the plant-associated persona(s), such as “Do you need water?”, and the corresponding natural-language output may be “Yes, I need water.”

In some embodiments, at block 512, the natural-language output can be modified based on a personality profile associated with the plant. For example, for a dehydrated plant associated with a sarcastic personality profile, the modified natural-language output can be “What do you think? Of course I need water!” Block 512 can include any features of block 206 of FIG. 2, block 308 of FIG. 3, and/or block 406 of FIG. 4, and vice versa.

In some embodiments, at block 514, the modified natural-language output can be provided to the user device. Block 514 can include any features of block 208 of FIG. 2, block 310 of FIG. 3, and/or block 408 of FIG. 4, and vice versa.

### “Learning Tree” Mode

According to some embodiments, “Learning Tree” mode can include methods of refining the analytical model that generates the insights for the “Talking Tree” mode. Method 300 and method 400 are examples of “Learning Tree” mode. Method 300 can comprise a method of refining the analytical model based on user responses to unexpected sensor measurements. Likewise, method 400 can comprise a method of refining the analytical model based on user feedback.

FIG. 3 depicts a simplified flowchart of a system-initiated method 300 for modifying an analytical model based on receiving, from a user device in a natural-language format, information regarding an unexpected sensor measurement, in accordance with some embodiments. At block 302, a set of sensor data associated with a plant can be received. At block 304, one or more sensor measurements that deviate from one or more predicted measurements generated by an analytical model can be identified from the set of sensor data. At block 306, a natural-language output pertaining to the deviation of the one or more sensor measurements from the one or more predicted measurements can be generated by the predicted model. At block 308, the natural-language output can be modified based on a personality profile associated with the plant. At block 310, the modified natural-language output, comprising a request for one or more responses from the user device, can be provided to the user device. At block 312, the one or more responses from the user device may be received based on the request. At block 314, the analytical model can be modified based on the one or more responses received from the user device. Method 300 can be initiated by the system based on information regarding an unexpected sensor measurement among the received sensor data. User input is not needed to initiate method 300, but user responses can be used to determine appropriate modifications to the analytical model. For example, when the plant is experiencing unexpectedly fast growth, the system can ask the user “Did you apply fertilizer recently?” If the user responds with “Yes,” the system can ask follow-up questions such as “When did you apply fertilizer?”, “What type of fertilizer did you apply?”, or “How much fertilizer did you apply?” The user can continue the conversation by responding to the follow-up questions, and the user's responses can be used to modify the analytical model to account for the type, timing, and amount of fertilizer application. In this way, user interventions with the plant can be reconciled against the analytical model to determine whether the user interventions are responsible for deviations between predicted and observed measurements. In some embodiments, one or more components of a sensor of the present disclosure can be programmed to trigger an alarm, alert, or other notification when a measurement exceeds a predetermined threshold value and/or deviates from one or more predicted measurements generated by an analytical model of the present disclosure. In some embodiments, a processor of the present disclosure can be programmed to trigger an alarm, alert, or other notification when a measurement obtained by one or more components of the sensor exceeds a predetermined threshold value and/or deviates from one or more predicted measurements generated by an analytical model of the present disclosure. For example, the sensor can trigger an alarm, alert, or other notification when tree lean exceeds a predetermined threshold lean value based on data from an accelerometer, indicating that the tree or plant part is at risk of falling.

In some embodiments, method 300 can be performed, for example, using one or more electronic devices implementing a software platform. In an exemplary example, method 300 can be performed by system 100. In some examples, method 300 can be performed using a client-server system, and the blocks of method 300 can be divided up in any manner between a server and one or more client devices (i.e., user devices). In other examples, method 300 can be performed using only a client device or only multiple client devices. Thus, while portions of method 300 are described herein as being performed by particular devices of a client-server system, it will be appreciated that method 300 is not so limited. In method 300, some blocks can, optionally, be combined, the order of some blocks can, optionally, be changed, and some blocks can, optionally, be omitted. In some examples, additional steps can be performed in combination with the method 300. Accordingly, the operations as illustrated (and described in greater detail below) are exemplary by nature and, as such, should not be viewed as limiting.

In some embodiments, at block 302, a set of sensor data associated with a plant can be received. Block 302 can include any features of block 202 of FIG. 2, block 402 of FIG. 4, and/or block 502 of FIG. 5, and vice versa.

In some embodiments, at block 304, one or more sensor measurements that deviate from one or more predicted measurements generated by an analytical model can be identified from the set of sensor data. For example, the processors may identify sensor measurements that indicate that a plant is growing abnormally fast compared to the predictions of the analytical model.

In some embodiments, at block 306, a natural-language output pertaining to the deviation of the one or more sensor measurements from the one or more predicted measurements can be generated by the predicted model. Block 306 can include any features of block 204 of FIG. 2, block 404 of FIG. 4, and/or blocks 504-510 of FIG. 5, and vice versa.

In some embodiments, at block 308, the natural-language output can be modified based on a personality profile associated with the plant. For example, for a plant that is associated with a friendly personality profile, the modified natural-language output can be “Hey there! I'm happy to say that I've been growing quickly. Did you apply fertilizer to me recently?” Block 308 can include any features of block 206 of FIG. 2, block 406 of FIG. 4, and/or block 512 of FIG. 5, and vice versa.

In some embodiments, at block 310, the modified natural-language output, comprising a request for one or more responses from the user device, can be provided to the user device. The request for one or more responses from the user device can include, but are not limited to, a text box, a predefined response button, an option for uploading a file, or any combination thereof. For example, when the request is a yes/no question, a “yes” response button and a “no” response button can appear on the display screen of the user device. Block 310 can include any features of block 208 of FIG. 2, block 408 of FIG. 4, and/or block 514 of FIG. 5, and vice versa.

In some embodiments, at block 312, the one or more responses from the user device may be received based on the request. For example, when a user selects the “yes” response button, the system can receive the user's selected response. Block 312 can comprise any features of block 410 of FIG. 4, and vice versa.

In some embodiments, at block 314, the analytical model can be modified based on the one or more responses received from the user device. For example, when the plant is experiencing unexpectedly fast growth, the system can ask the user “Did you apply fertilizer recently?” If the user responds with “Yes,” the system can ask follow-up questions such as “When did you apply fertilizer?”, “What type of fertilizer did you apply?”, or “How much fertilizer did you apply?” The user can continue the conversation by responding to the follow-up questions, and the user's responses can be used to modify the analytical model to account for the type, timing, and amount of fertilizer application. Block 314 can comprise any features of block 412 of FIG. 4, and vice versa.

FIG. 4 depicts a simplified flowchart of a system-initiated method 400 for modifying an analytical model based on receiving feedback from a user device in a natural-language format, in accordance with some embodiments. At block 402, a set of sensor data associated with a plant can be received. At block 404, a natural-language output can be generated based on the set of sensor data. At block 406, a natural-language output can be modified based on a personality profile associated with the plant. At block 408, the modified natural-language output, comprising a request for feedback from the user device, can be provided to the user device. At block 410, feedback can be received from the user device based on the request. At block 412, the analytical model can be modified based on the feedback received from the user device. Method 400 can be initiated by the system at any time. Information regarding an unexpected sensor measurement among the received sensor data is not needed to initiate method 400. Likewise, user input is not needed to initiate method 400, but user feedback can be used to determine appropriate modifications to the analytical model. For example, every 14 days, the system can request user feedback regarding the quality of the guidance, alerts, notifications, or answers to questions, and the user's feedback can be used to modify the analytical model.

In some embodiments, method 400 can be performed, for example, using one or more electronic devices implementing a software platform. In an exemplary example, method 400 can be performed by system 100. In some examples, method 400 can be performed using a client-server system, and the blocks of method 400 can be divided up in any manner between a server and one or more client devices (i.e., user devices). In other examples, method 400 can be performed using only a client device or only multiple client devices. Thus, while portions of method 400 are described herein as being performed by particular devices of a client-server system, it will be appreciated that method 400 is not so limited. In method 400, some blocks can, optionally, be combined, the order of some blocks can, optionally, be changed, and some blocks can, optionally, be omitted. In some examples, additional steps can be performed in combination with the method 400. Accordingly, the operations as illustrated (and described in greater detail below) are exemplary by nature and, as such, should not be viewed as limiting.

In some embodiments, at block 402, a set of sensor data associated with a plant can be received. Block 402 can include any features of block 202 of FIG. 2, block 302 of FIG. 3, and/or block 502 of FIG. 5, and vice versa.

In some embodiments, at block 404, a natural-language output can be generated based on the set of sensor data. Block 404 can include any features of block 204 of FIG. 2, block 306 of FIG. 3, and/or blocks 504-510 of FIG. 5, and vice versa.

In some embodiments, at block 406 a natural-language output can be modified based on a personality profile associated with the plant. Block 406 can include any features of block 206 of FIG. 2, block 308 of FIG. 3, and/or block 512 of FIG. 5, and vice versa.

In some embodiments, at block 408, the modified natural-language output, comprising a request for feedback from the user device, can be provided to the user device. For example, the request for feedback can be a message such as “Hi there, I'm Zesty the lemon tree. Have my messages to you been useful so far?” Block 408 can include any features of block 208 of FIG. 2, block 310 of FIG. 3, and/or block 514 of FIG. 5, and vice versa.

In some embodiments, at block 410, feedback can be received from the user device based on the request. For example, in response to a question from the system about the quality of the system's insights, the user can respond “Your messages have been useful, but I'd like to receive them more frequently.” Block 410 can comprise any features of block 312 of FIG. 3, and vice versa.

In some embodiments, at block 412, the analytical model can be modified based on the feedback received from the user device. For example, upon receiving feedback that the user would like to receive more frequent messages, the processors can instruct the analytical model to generate insights on a twice-daily basis instead of a once-daily basis. Block 412 can comprise any features of block 314 of FIG. 3, and vice versa.

The foregoing description sets forth exemplary systems, methods, parameters and the like. It should be recognized, however, that such description is not intended as a limitation on the scope of the present disclosure but is instead provided as a description of exemplary embodiments. The illustrative embodiments described above are not intended to be exhaustive or to limit the disclosure to the precise forms disclosed. Many modifications and variations are possible in view of the above teachings. The embodiments were chosen and described to best explain the principles of the disclosed techniques and their practical applications. Others skilled in the art are thereby enabled to best utilize the techniques and various embodiments with various modifications as are suited to the particular use contemplated.

### EXAMPLES

The presently disclosed subject matter will be better understood by reference to the following Examples, which are provided as exemplary of the invention, and not by way of limitation.

### Example 1: “Talking Tree” Mode Interaction with Zesty the Lemon Tree

FIG. 8 depicts an exemplary interaction between the user and the plant-associated persona(s). In this example, the user is receiving a notification based on sensor data from dendrometers on a lemon tree. This sensor data is used to create a digital persona, or personality profile, of the tree. The digital persona of the tree can name itself and has selected “Zesty” as a suitable name. FIG. 8 shows how a graphical representation or avatar can be updated, e.g., based at least in part on the type of plant associated with sensor(s), aspects of the personality profile for the plant, sensor data associated with the plant, etc. In this example, the avatar for the lemon tree (“Zesty”) in the left panel is green and healthy when sensor data indicate the plant is healthy, but less vibrant and shriveled in appearance in the right panel when sensor data indicate the plant needs water. As such, the graphical representation or avatar can convey complex concepts or plant needs in a readily understandable way via updates to its appearance.

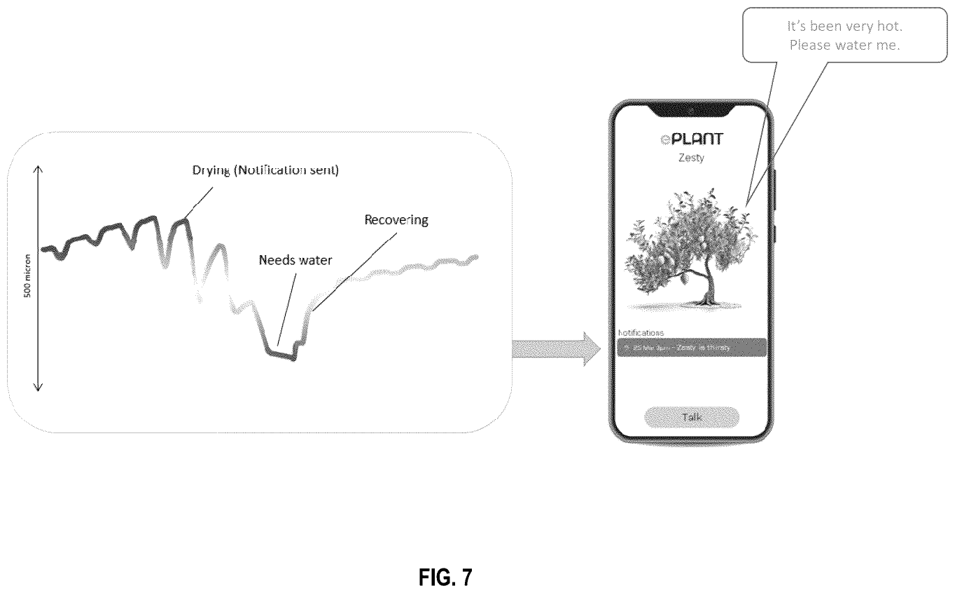

As shown in the left panel of FIG. 7, the sensor data shows that the shrinkage in the tree's size exceeds normal daily parameters, indicating that the tree is experiencing dehydration, which is an undesirable change in the tree's health status. Accordingly, the system displays a notification on the user device, which reads “Zesty is thirsty.” The system also displays a message from the perspective of Zesty, which reads “It's been very hot. Please water me”, as shown in the right panel of FIG. 7. A graphical representation or avatar associated with Zesty can also be updated to indicate changes in health status, as shown in the right panel of FIG. 8. Notifications and messages can appear within the communication application on the user's device, outside of the communication application, or both. Upon being watered by the user, the sensor data shows that the tree is recovering from dehydration. At this time, the system can send notifications and messages updating the user of the tree's new health status. In some embodiments, once the dendrometer data indicates that the tree is recovering, the system can display a notification on the user device asking if the user watered the tree. This helps the system improve the analytical model(s) by ascertaining whether a user intervention with the plant (e.g., applying water or fertilizer, pruning, etc.) has occurred and reconciling the change in plant measurement(s) with the nature of the intervention(s).

### Example 2: Exemplary Mobile Application Graphical User Interface (GUI)

FIG. 8 depicts an exemplary GUI of the mobile application for communicating with plant-associated persona(s). In some embodiments, the plant and its health can be represented by one or more of a name, graphical icon, and picture. The GUI can allow the user to toggle between displaying one of multiple plants associated with the user device, as well as between public or shared trees that have been made available to other users of the mobile application. This display can be in map view or list view. For example, a user can select between viewing his or her lemon tree, a neighbor's oak tree, or a publicly viewable pine tree by pressing their respective icons on a location map within the application. Furthermore, the user can toggle between displaying plants individually or as a collective. For example, a user who owns an orchard of trees can toggle between communicating with a tree personality that represents an individual lemon tree or a caretaker personality that represents the entire orchard. Using the GUI, a user can select an individual plant (or a collective of plants) to engage in a chat dialog. The chat dialog can be in graphical, visual, or audio formats. For example, the GUI can provide users with input options for typing, speaking, or using text-to-speech to chat with the plant. During the chat dialog, the user can be prompted to respond to various messages from the system. For example, the GUI may display “agree,” “disagree,” or “skip” response buttons for the user to provide feedback on the insights of the system. The GUI may also display sliders, multiple choice options, or text boxes to allow the user to provide feedback directly to the system. Notifications from the system may appear on the GUI as pop-up messages in line, such as standard pop-up messages on Android or iOS devices.

In this example, the plant is Zesty, the lemon tree of Example 1. As shown in FIG. 8, on the left screen, Zesty is depicted as a graphical icon of a healthy, well-watered lemon tree. The GUI is displaying a message from Zesty about its health status, which reads “I'm healthy and happy!” Conversely, as shown in FIG. 8, on the right screen, Zesty is depicted as a sickly, dehydrated lemon tree. As such, the GUI is displaying a notification that Zesty is thirsty, as well as a message from Zesty which reads “It's been very hot. Please water me.” The user can press the “Talk” button to communicate with a digital representation of Zesty, such as asking follow-up questions about how much water is needed or for how long Zesty has been experiencing dehydration.

### Example 3: Obtaining Plant Health Data from Dendrometer Measurements

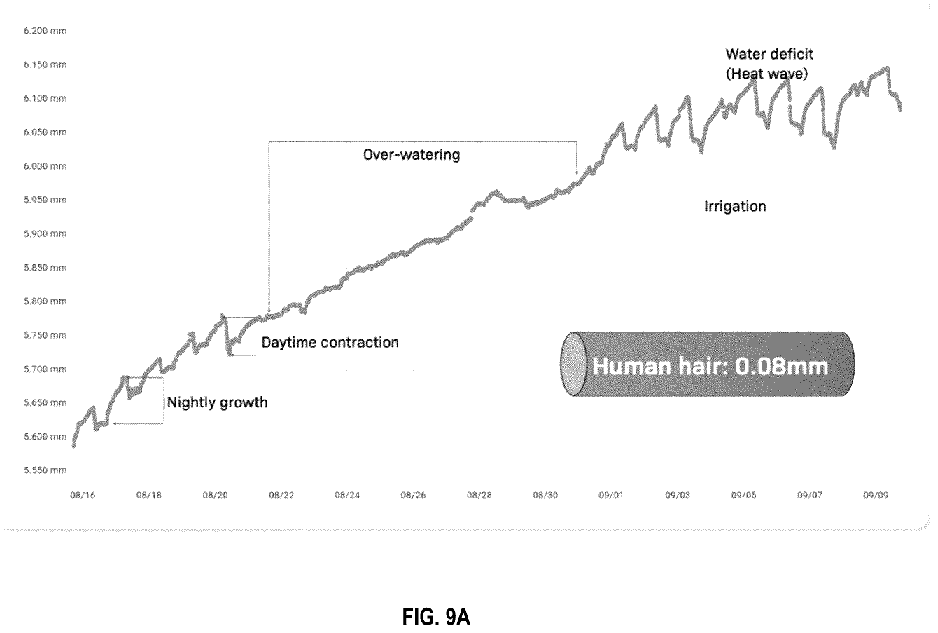

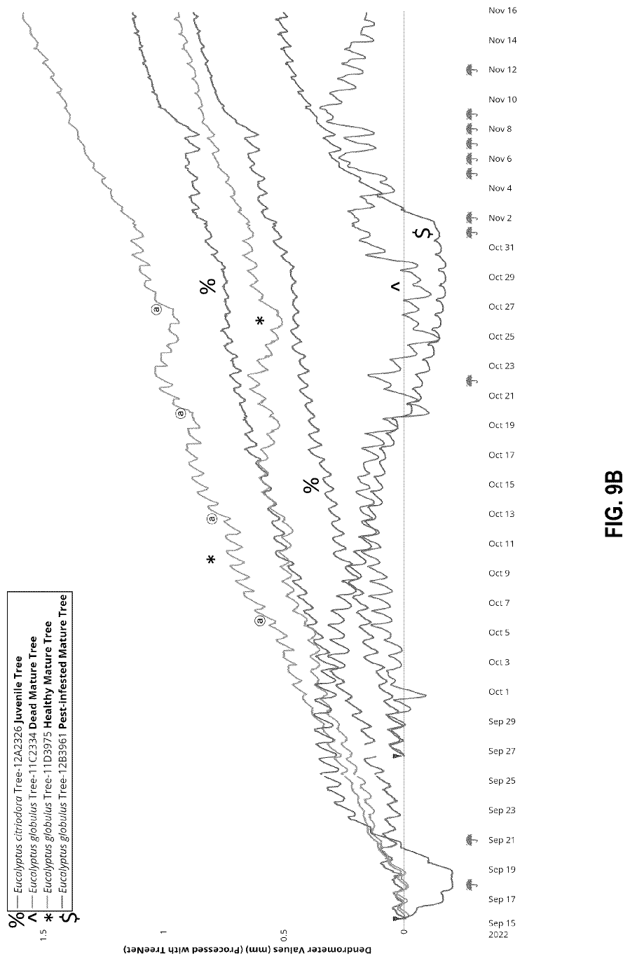

FIGS. 9A and 9B depict exemplary graphs of dendrometer measurements associated with plant health, in accordance with some embodiments. In this example, the dendrometers are attached to various types of trees and are configured to record size measurements of the trees over a given period of time.

FIG. 9A depicts the changes in size of a single tree over a three-week period. The tree size measurements fluctuate up and down on a daily basis due to nightly growth and daytime contraction. Size measurements are shown on the y-axis at a scale with increments less than 0.050 mm, showing the resolution achieved with the sensors described herein. During a period of regular health, the tree size measurements generally trend upwards in a linear manner. During a period of over-watering, the tree size measurements do not fluctuate as much and exhibit only the linear upward growth pattern. During a period of water deficit, the tree size measurements do not exhibit the linear upward growth pattern and only exhibits the daily fluctuations in size. In some embodiments, the systems described herein can recognize these patterns from the dendrometer measurements as “appropriate watering,” “overwatering,” and “underwatering” and inform the user accordingly.

FIG. 9B depicts the changes in size of four types of trees (juvenile, dead mature, healthy mature, and pest-infected mature trees) over a three-month period. In this example, the juvenile trees exhibit a faster rate of growth than the healthy mature trees, as depicted by their tree size measurements trending linearly upwards with greater slopes than those of the healthy mature trees. While both the juvenile trees and the healthy mature trees experience significant growth over the three-month period, the dead mature trees and pest-infected mature trees do not. In some embodiments, the systems described herein can recognize these patterns from the dendrometer measurements and classify the health status of the tree accordingly.

1. A method for providing a natural-language interface for conversing with a persona associated with a plant, comprising, at one or more processors:
receiving a set of sensor data associated with the plant;
generating, via an analytical model, a natural-language output based on the set of sensor data;
modifying the natural-language output based on a personality profile associated with the persona; and
providing the modified natural-language output to a user device.

2. A method for providing a natural-language interface for conversing with a persona associated with a plant, comprising, at one or more processors:
receiving a set of sensor data associated with the plant;
generating, via an analytical model, a data product based on the set of sensor data;
generating one or more prompts for a large language model based on one or more of:
the data product,
user data,
plant data,
environmental data,
a personality profile associated with the persona, and
data entered by a user;
providing the large language model with the one or more prompts;
receiving a natural-language output from the large language model in response to the one or more prompts;
modifying the natural-language output based on the personality profile associated with the persona; and
providing the modified natural-language output to a user device.

3. The method of claim 1, further comprising: receiving, from the user device, a natural-language user request associated with the plant, wherein the natural-language user request is received in one or more of audio, text, and graphical format.

4. (canceled)

5. The method of claim 3, wherein generating the natural-language output comprises:
converting the natural-language user request into machine-language instructions;
based on the machine-language instructions, inputting a selection from the set of sensor data into the analytical model;
receiving a machine-language output from the analytical model; and
converting the machine-language output into the natural-language output.

6. The method of claim 3, wherein the natural-language output is generated based on both the natural-language user request and the set of sensor data.

7. The method of claim 3, wherein the modified natural-language output comprises a response to the natural-language user request according to the personal profile associated with the persona.

8. (canceled)

9. The method of claim 1, wherein the set of sensor data comprises one or more sensor measurements that deviate from one or more predicted measurements generated by the analytical model, and wherein the personality profile associated with the persona is configured to be modified based on a deviation of the one or more sensor measurements from the one or more predicted measurements.

10-11. (canceled)

12. The method of claim 1, wherein the modified natural-language output comprises a request for feedback pertaining to the analytical model, and wherein the analytical model is modified based on one or more responses from the user device addressing the request for feedback.

13-14. (canceled)

15. The method of claim 1, wherein the set of sensor data is obtained from one or more sensors associated with the plant, and wherein the one or more sensors comprise:
a) one or more fasteners configured to be positioned in or around a part of the associated plant;
b) two or more components selected from the group consisting of: a dendrometer, an accelerometer, an air temperature sensor, a humidity sensor, and a light sensor;
c) a processor; and
d) a power supply.

16-20. (canceled)

21. The method of claim 1, wherein the set of sensor data comprises data on one or more of air temperature, humidity, light, hydration, plant movement, and plant dimensions.

22. The method of claim 1, wherein the set of sensor data is processed by one or more of: the analytical model, wherein the analytical model comprises one or more of a linear model, a temporal fusion transformer, a neural network, a heuristic model, and a decision tree; a machine-learning algorithm; and an artificial intelligence program.

23. (canceled)

24. The method of claim 22, wherein the analytical model is trained on one or more of sensor measurements, plant data, and environmental data.

25. The method of claim 1, wherein modifying the natural-language output comprises:
inputting the natural-language output into a large language model, wherein the large language model is trained on plant data;
instructing the large language model to modify the natural-language output based on the personality profile; and
receiving the modified natural-language output from the large language model.

26-27. (canceled)

28. The method of claim 1, wherein generating the natural-language output comprises:
inputting a selection from the set of sensor data into the analytical model;
receiving a machine-language output from the analytical model; and
converting the machine-language output into the natural-language output.

29. The method of claim 1, further comprising: modifying the natural-language output based on a user profile associated with the user.

30. (canceled)

31. The method of claim 1, wherein the modified natural-language output comprises one or more of: a suggested intervention for the plant; information pertaining to plant health; and a visual presentation of one or more of sensor measurement(s), plant data, and environmental data.

32-33. (canceled)

34. The method of claim 1, wherein the personality profile associated with the persona comprises one or more instructions configured to refine the large language model.

35. The method of claim 1, wherein the personality profile associated with the persona is configured to be modified based on the growth rate and/or transpiration rate of the plant.

36. The method of claim 1, further comprising: providing instructions for displaying a graphical representation of the plant at the user device, wherein the graphical representation of the plant is based, at least in part, on a personality profile associated with the plant, and wherein the graphical representation of the plant is configured to be modified based, at least in part, on the set of sensor data associated with the plant.

37-38. (canceled)

39. The method of claim 1, wherein the persona is associated with multiple plants.

40-54. (canceled)
